(19) United States
(12) Patent Application Publication
(10) Pub. No.: US 2021/0401613 A1
(43) Pub. Date: Dec. 30, 2021

(54) MALE EXTERNAL URINE COLLECTION DEVICE

(71) Applicant: CHIH-TA CHIANG, New Taipei City (TW)

(72) Inventor: CHIH-TA CHIANG, New Taipei City (TW)

(21) Appl. No.: 17/330,429

(22) Filed: May 26, 2021

(30) Foreign Application Priority Data
Jun. 24, 2020 (TW) 109121562

(51) Int. Cl. A61F 5/453; A61F 5/44

(52) U.S. Cl. CPC A61F 5/453; A61F 5/4408; A61F 5/4405

(57) ABSTRACT

A male external urine collection device includes an inner member and an external member. The inner member includes an upper sleeve with a first opening and a second opening respectively at the upper sleeve's two ends, a ring encircling an opening and fringing the first opening, and a lower mesh joined to the second opening. The external member accommodates the upper sleeve and the lower mesh, where the upper sleeve and the lower mesh are threaded through a first end of the external member, and the external member has an outlet at a second end opposite to the first end for urine collection.

### BACKGROUND OF THE INVENTION

### (a) Technical Field of the Invention

The present invention is generally related to sanitation devices, and more particular to a male external urine collection device for the disabled, demented, physically confined, or physiologically dysfunctional.

### (b) Description of the Prior Art

Urination is one of human's major body functions. As people age and the physiological function deteriorates, or as people are injured, become dementia or disabled, they can no longer control urination by themselves. They need constant care and often have to wear paper diapers. Paper diapers are expensive, not reusable, and airtight. The wearers may suffer eczema, rash, or other skin disease. Constantly replacing diapers is also troublesome and costly. The used diapers increase waste and are not environmentally friendly.

Urinary catheters may also be applied to collect urine into a urine bag so that paper diapers are not required. However, catheter leakage, blockage, or falling off may happen if the caretaker is not careful or when the patient turns over. Urinary catheters also require constant replacement by professional medical personnel to avoid urethral inflammation or infection. These all bring extra burden to the caretaker or the patient's family.

### SUMMARY OF THE INVENTION

Therefore, a novel male external urine collection device is provided herein. The present invention involves the following design ideas. Firstly, using elastic mesh to tightly wrap and enclose the urine organ so that, if it is more expanded, the wrapping or enclosure effect is even more reliable. A meshed opening is provided at the bottom and connected with a transmission tube to facilitate urine collection. Secondly, the male external urine collection device may be integrated with a lower-body wear for reliable positioning. Thirdly, the male external urine collection device is designed following the idea of medical double check valve to prevent infection resulted from urine reflow. The male external urine collection device may be applied with traditional catheters for disabled, demented, physically confined, or physiologically dysfunctional male users. The present invention has various advantages such as infection prevention, leakage proof, reflow avoidance, etc.

A major objective of the present invention is to provide a male external urine collection device that includes an inner member and an external member. The inner member includes an upper sleeve with a first opening and a second opening respectively at the upper sleeve's two ends, a ring encircling an opening and fringing the first opening, and a lower mesh joined to the second opening. The external member accommodates the upper sleeve and the lower mesh, where the upper sleeve and the lower mesh are threaded through a first end of the external member, and the external member has an outlet at a second end opposite to the first end for urine collection.

A second objective of the present invention is to provide a male external urine collection device where the upper sleeve is made of a silicone, rubber, or plastic material into a thin tubular shape or an elastic tubular mesh. The upper sleeve has a number of protrusions on an outer circumference, and each protrusion has an inversed V shape, a H shape, a #shape, a X shape, or a Y shape.

A third objective of the present invention is to provide a male external urine collection device that includes a lower-body wear, an inner member, and an external member. A through hole is provided in a lower part of the lower-body wear a first fastener of the inner member is exposed out of the through hole and coupled to a second fastener of the external member so that the inner member and the external member are integrated together across the through hole. The second member may be connected to a urine bag or an anti-reflow unit to facilitate urine collection and to prevent urine reflow and infection.

A fourth objective of the present invention is to provide a male external urine collection device that includes a lower-body wear worn on a male user's lower body. The lower-body wear may be an underpants, a paper pants, a shorts, or a paper diaper. The lower-body wear has a through hole in a lower part of the lower-body wear.

A fifth objective of the present invention is to provide a male external urine collection device including an inner member and an external member. The internal member includes an upper sleeve and a lower mesh made of a silicon, plastic, or rubber material into a thin tube or elastic tubular mesh. The inner member and an external member are coupled together through matched threads, matched protrusions and notches, etc. The second member may be made of a silicon, plastic, or rubber material into a tubular shape. The external member may be connected to an anti-reflow unit, an on-off valve, an urine bag, an urine collection bucket, etc.

A sixth objective of the present invention is to provide a male external urine collection device that can also be applied to the transmission of various kinds of liquids, gases, or fluids. The opening of the inner member may be connected to an air duct or a water pipe which is further covered by the sleeve to achieve high tightness and no leakage transmission for liquids or gases.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective diagram showing a male external urine collection device according to a first embodiment of the present invention.

FIG. 2 is a perspective diagram showing an application scenario of the male external urine collection device of FIG. 1.

FIG. 3 is a perspective diagram showing another application scenario of the male external urine collection device of FIG. 1.

FIG. 4 is a perspective diagram showing a male external urine collection device according to a second embodiment of the present invention.

FIG. 5 is a perspective diagram showing an application scenario of the male external urine collection device of FIG. 4.

FIG. 6 is a perspective diagram showing the application scenario of the male external urine collection device of FIG. 5 after assembly.

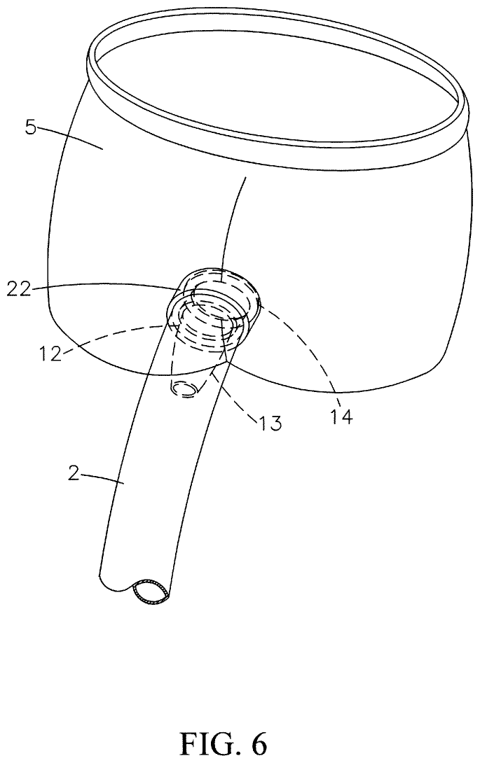

FIG. 7 is a schematic profile diagram showing the application scenario of the male external urine collection device of FIG. 6.

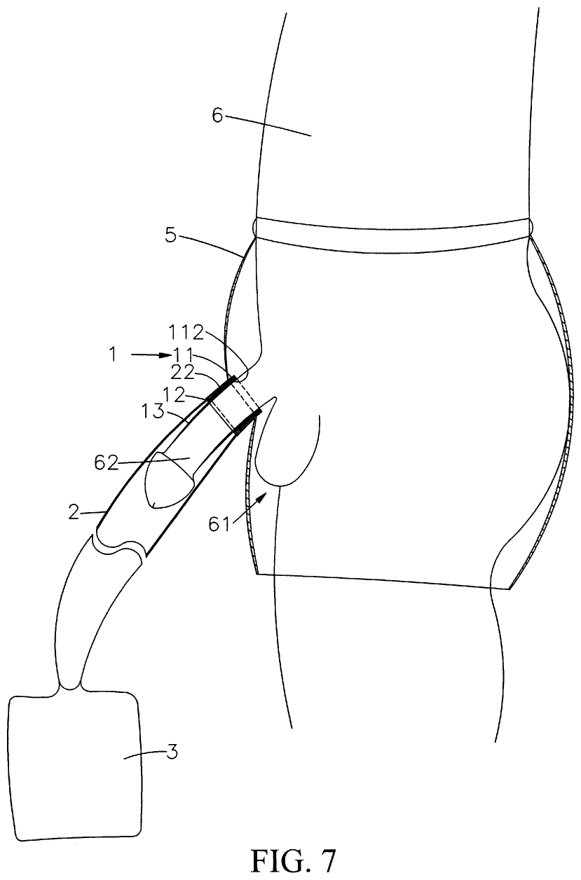

FIG. 8 is a perspective diagram showing another application scenario of the male external urine collection device of FIG. 4.

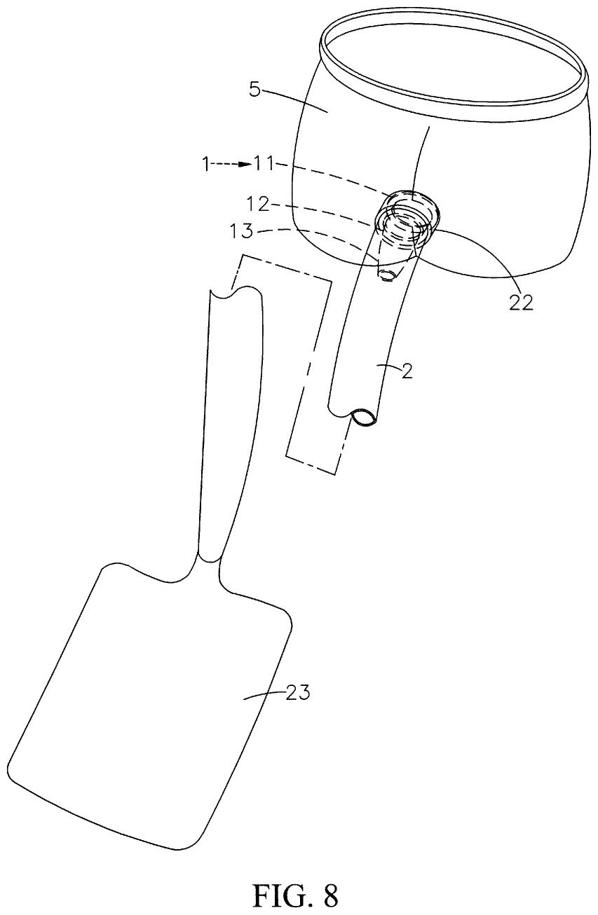

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

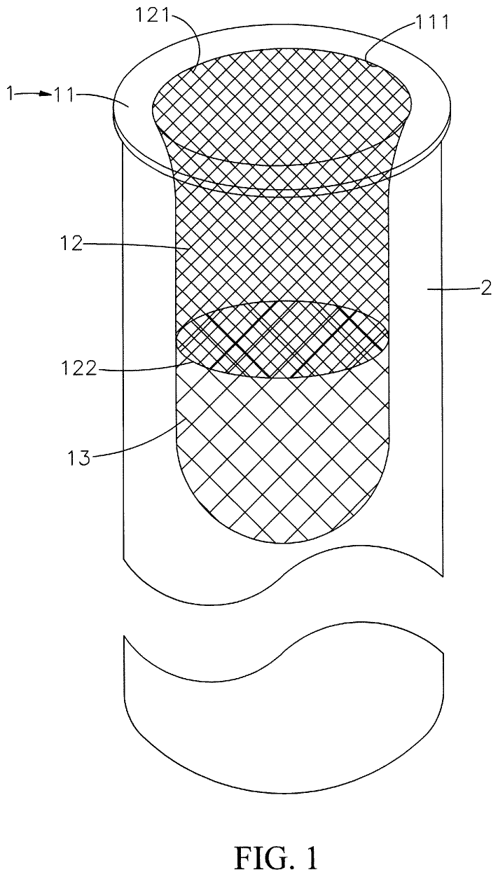

As shown in FIG. 1, a male external urine collection device according to a first embodiment of the present invention includes an inner member 1 and an external member 2.

The tubular inner member 1 includes an upper sleeve 12 with a first opening 121 and a second opening 122 respectively at the upper sleeve 12's two ends, a ring 11 encircling an opening 111 and fringing the first opening 121, and a lower mesh 13 joined to the second opening 122.

The upper sleeve 12 is made of a silicone, rubber, or plastic material into a thin tubular shape or into an elastic tubular mesh. The upper sleeve 12 may have a number of protrusions on an outer circumference, each having an inversed V shape, a H shape, a #shape, a X shape, or a Y shape.

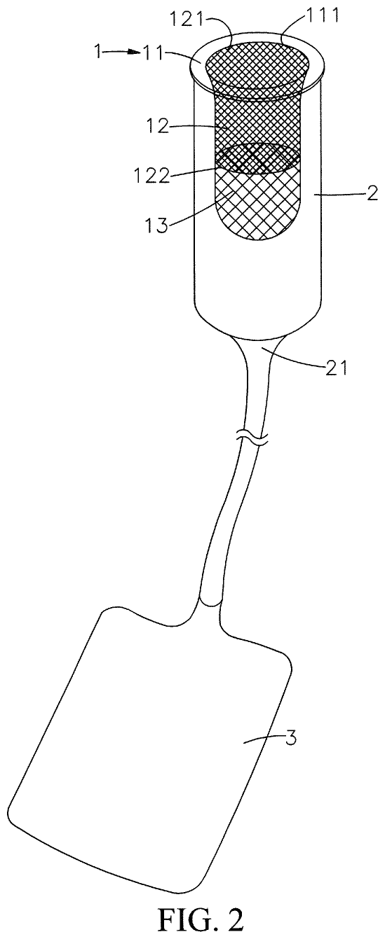

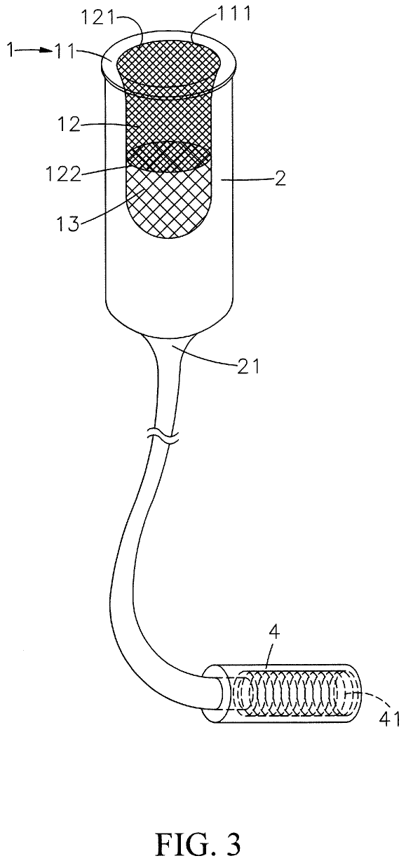

The upper sleeve 12 and the lower mesh 13, after being joined together, are threaded into an end of the external member 2. The external member 2 has an outlet 21 at another end opposite to the inner member 1. As shown in FIGS. 2 and 3, a urine bag 3 and/or an anti-reflow unit 4 may be connected to the outlet 21 through a transmission tube (not numbered). The anti-reflow unit 4 includes an elastic element 41, and prevents urine collected in the urine bag 3 from flowing back to the external member 2 through the pressure difference between the external member 2 and the urine bag 3. The anti-reflow unit 4 also prevents the inner member 1 and the external member 2 from being contaminated by bacteria in the urine bag 3. The transmission tube is made of a silicone, plastic, or rubber material.

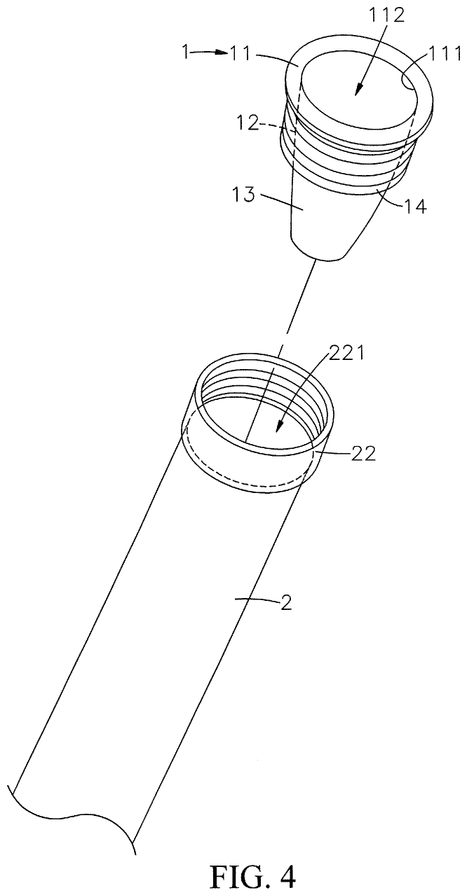

As shown in FIG. 4, a male external urine collection device according to a second embodiment of the present invention includes an inner member 1, an external member 2, and a lower-body wear 5.

The tubular inner member 1 includes an upper sleeve 12, a ring 11 encircling an opening 111 and fringing a first opening of the upper sleeve 12, a lower mesh 13 joined to a second opening of the upper sleeve 12, and a first fastener 14 surrounding the upper sleeve 12 and the lower mesh 13. Alternatively, the ring 11 and first fastener 14 are vertically joined together to form an accommodation space 112, and the flexible upper sleeve 12 and lower mesh 13 are embedded in or integrally formed with the accommodation space 112.

The external member 2 has a second fastener 22 at a first end and an outlet 21 at another end. A through channel 221 runs from the second fastener 22 to the outlet 21.

The lower-body wear 5 has a through hole 51 in a lower part of the lower-body wear.

The inner member 1's ring 11 has an outer diameter larger than the through hole 51's aperture. The first fastener 14 has an outer diameter equal or smaller than the through hole 51's aperture.

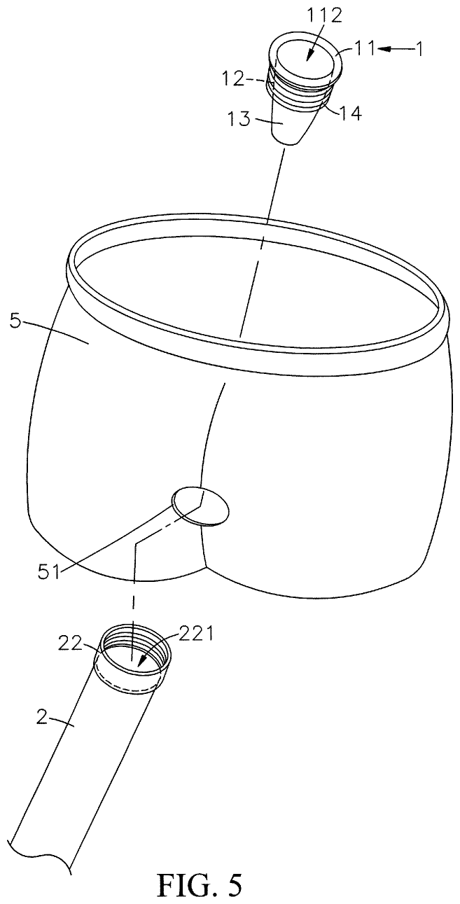

As shown in FIG. 5, to apply the male external urine collection device, the inner member 1 is run inside out through the through hole 51 of the lower-body wear 5 so that the ring 11 is blocked by the through hole 51 and the upper sleeve 12, the lower mesh 13, and the first fastener 14 are exposed outside the through hole 51. The external member 2's second fastener 22 is then coupled to the first fastener 14 and the upper sleeve 12 and the lower mesh 13 are extended into the through channel 221 inside the external member 2. As such, the inner member 1 and the external member 2 are joined together across the through hole 51 of the lower-body wear 5.

The inner member 1's first fastener 14 is configured with external threads while the external member 2's second fastener 22 is configured with matched internal threads, so that they may be reliably coupled together. Alternatively, protrusions (or notches) may be configured around the outer circumference of the first fastener 14 and, correspondingly, notches (or protrusions) may be configured along an inner wall of the through channel 221 of the second fastener 22. It is also possible to have internal threads around the accommodation space 112 and external threads around the outer circumference of the second fastener 22. Furthermore, it is also possible to have protrusions (or notches) configured in the accommodation space 112 and notches (protrusions) on the outer circumference of the second fastener 22.

When the inner member 1, external member 2 and the lower-body wear 5 are put to use, the lower-body wear 5 may be an underpants, a paper pants, a shorts, or a paper diaper for wearing on the lower body 61 of a disabled, demented, physically confined, or physiologically dysfunctional male user 6. The urinary organ 62 of the male user 6 is threaded into the accommodation space 112 of the inner member 1 through the ring 11. The elastic sleeve 12 and lower mesh 13 then fit, wrap, and reliably position around the urinary organ 62. The urinary organ 62, together with the sleeve 12 and lower mesh 13, enters the through channel 221 of the external member 2's second fastener 22. The external member 22 may be made of a silicone, plastic, or rubber material. An on-off valve, a urine bag 3, or a urine collection bucket may be connected to the outlet 21 of the external member 22.

When the male user 6 urinates, urine will flow through the external member 2 into the urine bag 3. As the male user 6's urinary organ 62 is confined by the sleeve 12 and lower mesh 13 and does not break away from the lower-body wear 5, the inner member 1, the external member 2. In addition, as the inner member 1 and the external member 2 also prevents urine from reflowing and leakage, the male user 6's lower body 61 would be kept dry and clean. The male user 6 would not suffer eczema, rash, or other skin disease. The urine bag 3 may be conveniently detached from the outlet 21 and then emptied, cleaned, disinfected, and sterilized. The inner member 1 and the external member 2 may be assembled or disassembled through the through hole 51 of the lower-body wear 5. The inner member 1 and the external member 2 therefore can be disinfected and sterilized before and after usage.

Furthermore, the male external urine collection device can also be applied to the transmission of various kinds of liquids, gases, or fluids. The opening 111 of the inner member 1 may be connected to an air duct or a water pipe (not shown) which is further covered by the sleeve 12 to achieve high tightness and no leakage transmission for liquids or gases.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A male external urine collection device, comprising an inner member and an external member, wherein
the inner member comprises an upper sleeve with a first opening and a second opening respectively at the upper sleeve's two ends, a ring encircling an opening and fringing the first opening, and a lower mesh joined to the second opening; and
the external member accommodating the upper sleeve and the lower mesh, where the upper sleeve and the lower mesh are threaded through a first end of the external member, and the external member has an outlet at a second end opposite to the first end.

2. The male external urine collection device according to claim 1, wherein the upper sleeve is made of a silicone, rubber, or plastic material into a thin tubular shape or an elastic tubular mesh.

3. The male external urine collection device according to claim 2, wherein the upper sleeve has a plurality of protrusions on an outer circumference; each protrusion has an inversed V shape, a H shape, a #shape, a X shape, or a Y shape.

4. The male external urine collection device according to claim 1, wherein the inner member further comprises a first fastener surrounding the upper sleeve and the lower mesh; and the external member has a second fastener at the first end of the external member for coupling with the first fastener.

5. The male external urine collection device according to claim 4, wherein the first fastener and the second fastener are matched external and internal threads, or matched protrusion and notch, respectively.

6. A male external urine collection device, comprising a lower-body wear, an inner member and an external member, wherein
the lower-body wear has a through hole in a lower part of the lower-body wear;
the inner member comprises an upper sleeve with a first opening and a second opening respectively at the upper sleeve's two ends, a ring encircling an opening and fringing the first opening, a lower mesh joined to the second opening, and a first fastener vertically joined to the ring to form an accommodation space where the upper sleeve and the lower mesh are embedded in the accommodation space;
the external member having a first end, a second end opposite to the first end, a second fastener at the first end, an outlet at the second end, a through channel extending from the second fastener to the outlet, and a transmission tube connected to the outlet;
the inner member is run from inside the lower-body wear out through the through hole of the lower-body wear
the upper sleeve and the lower mesh are threaded through the first end of the external member so that the upper sleeve, the lower mesh, and the first fastener are exposed outside the through hole;
the external member's second fastener is coupled to the first fastener and the upper sleeve and the lower mesh are extended into the through channel inside the external member; and
the inner member and the external member are thereby joined together across the through hole of the lower-body wear.

7. The male external urine collection device according to claim 6, wherein the lower-body wear is an underpants, a paper pants, a shorts, or a paper diaper.

8. The male external urine collection device according to claim 6, wherein the inner member's ring has an outer diameter larger than the through hole's aperture; and the first fastener has an outer diameter equal or smaller than the through hole's aperture.

9. The male external urine collection device according to claim 6, wherein the transmission tube is made of a silicone, plastic, or rubber material; and an on-off valve, a urine bag, or a urine collection bucket is connected to the outlet.
